(19) United States
(12) Patent Application Publication
(10) Pub. No.: US 2023/0175033 A1
(43) Pub. Date: Jun. 8, 2023

(54) PRODUCTION OF JASMONATES IN FILAMENTOUS FUNGI

(71) Applicant: Sumitomo Chemical Company, Limited, Tokyo (JP)

(72) Inventors: Hui Chen, North Billerica, MA (US); Oliver Yu, Lexington, MA (US)

(73) Assignee: Sumitomo Chemical Company, Limited, Tokyo (JP)

(21) Appl. No.: 17/923,077

(22) PCT Filed: May 4, 2021
(86) PCT No.: PCT/US2021/030594

§ 371 Date: Nov. 3, 2022

Related U.S. Application Data
(60) Provisional application No. 63/019,429, filed on May 4, 2020.

(51) Int. Cl. C12P 31/00; C12N 1/14

(52) U.S. Cl. CPC C12P 31/00; C12N 1/145; C12N 2501/999; C12N 2501/305; C12N 2527/00

(57) ABSTRACT

The present invention relates to improved methods for producing jasmonates such as jasmonic acid and methyl jasmonate in filamentous fungi under shaking conditions, hence enabling scale-up manufacturing processes using conventional fermenters. Specifically, one or more fungal quorum sensing molecules and/or jasmonate production elicitors can be added in the nutrient medium during cultivation of the filamentous fungi to induce formation of favorable morphologies and jasmonate production.

### RELATED APPLICATIONS

This application is a national stage filing under 35 U.S.C. § 371 of International Patent Application No. PCT/US2021/030594, filed May 4, 2021, entitled “PRODUCTION OF JASMONATES IN FILAMENTOUS FUNGI”, which claims the benefit under 35 U.S.C. § 119(e) to U.S. Provisional Application No. 63/019,429, filed May 4, 2020, and entitled “PRODUCTION OF JASMONATES IN FILAMENTOUS FUNGI,” the entire contents of which is incorporated herein by reference.

### FIELD OF THE INVENTION

The field of the invention relates to the production of jasmonates in filamentous fungi such as Lasiodiplodia iranensis.

### BACKGROUND OF THE INVENTION

Jasmonates, which include jasmonic acid (JA), methyl jasmonate (MeJA), and other precursors and derivatives in the jasmonic acid biosynthetic pathway, are α-linolenic acid-derived compounds of great economic importance. They are a class of plant hormones that play a central in role in plant defenses against necrotrophic pathogens and herbivorous insects. They also are powerful elicitors that induce the biosynthesis of a large number of secondary metabolites such as caffeoylputrescine in tomato leaves. See e.g., Chen et al., Proc. Natl. Acad. Sci. USA, 102:19237-19242 (2005); Vijayan et al., Proc. Natl. Acad. Sci. USA, 95:7209-7214 (1998); and Chen et al., FEBS Lett. 580: 2540-2546 (2006).

Methyl jasmonate which gives an odor reminiscent of the floral heart of jasmine is used for its floral notes for peach, apricot, grape and other flavors. It has been classified as generally recognized as safe (GRAS) by the Flavor Extract Manufacturers Association since 1973. In addition, methyl jasmonate also has been shown to have great potential as a novel class of anticancer drugs. Specifically, by inducing cytochrome C release in the mitochondria of cancer cells, methyl jasmonate can kill cancer cells while not harming normal cells. See Rotem et al., Cancer Res., 65: 1984-1993 (2005).

Due to the importance of jasmonates in agriculture, flavor and fragrance industry, and potentially medicine, there is considerable interest in producing jasmonates at large scale. Although jasmonates can be synthesized by organic chemistry, consumer demands for “natural” flavors have generated a market for jasmonates produced by bio-based processes. More importantly, chemically synthesized jasmonates are a mixture of biologically active and inactive isomers, whereas bio-based jasmonates are dominated by the biologically active isomers. Unfortunately, similar to other plant hormones, both jasmonic acid and methyl jasmonate exist only in trace amounts in higher plants (e.g., less than 10 μg in one kg of induced fresh tomato leaves), thus preventing the exploitation of higher plants as a commercial source of jasmonates. See Chen et al., FEBS Lett. 580: 2540-2546 (2006).

In contrast, bioproduction processes using filamentous fungi such as Lasiodiplodia theobromae (synonyms include Botryodiplodia theobromae and Diplodia gossypina), Fusarium oxysporum, and Gibberella fujikuroi can synthesize a large amount of jasmonic acid (e.g. 1-1.5 g/L). See U.S. Pat. No. 6,333,180, and Eng et al., PLoS One, 11: e0167627. In fact, jasmonic acid as a natural product was first isolated from a culture of the fungus Lasiodiplodia theobromae in 1971. See Aldridge et al., J. Chem. Soc. C, pp. 1623-1627 (1971).

However, it was also found that jasmonic acid was produced only in stationary flask cultures or stationary tray cultures. On the other hand, large-scale fermentation manufacturing uses fermenters that operate with rocking or orbital shaking to maximize production.

Accordingly, there remains a need in the art for improved jasmonate bioproduction methods, specifically those that can be scalable and can achieve high production titers under agitation conditions.

### SUMMARY OF THE INVENTION

The present invention addresses the problem described above by using quorum sensing molecules and/or elicitors to induce jasmonate production in filamentous fungi. A correlation was found between the mycelial morphology of such filamentous fungi and the level of jasmonate production. Specifically, high levels of jasmonate production were observed only when filamentous fungi are able to form a mycelial mat but not when the filamentous fungi are in free-floating pellet forms or clump into mycelial particles. The use of quorum sensing molecules enables the formation of a mycelial mat even when the filamentous fungi are grown under shaking conditions (e.g., by agitation systems). Jasmonate production elicitors are small molecules capable of inducing or awaking a cryptic biosynthetic pathway. In this case, jasmonate production elicitors are selected for their ability to induce or awake a cryptic biosynthetic pathway involved in jasmonate production by filamentous fungi.

Accordingly, in one aspect, the present invention provides a method for producing one or more jasmonates (e.g., jasmonic acid and/or methyl jasmonate), where the method comprises cultivating a strain of filamentous fungus organism in a nutrient medium under agitation, and isolating a jasmonate product from the nutrient medium. Representative genera of filamentous fungi include Lasiodiplodia, Fusarium, and Gibberella. In various embodiments, the filamentous fungus organism can be selected from the group consisting of Lasiodiplodia iranensis, Lasiodiplodia theobromae, Fusarium oxysporum, and Gibberella fujikuroi. In a representative embodiment, the filamentous fungus organism is Lasiodiplodia iranensis DWH-2 deposited under CCTCC Deposit No. M2017288.

In various embodiments, the nutrient medium can include at least one fungal quorum sensing molecule. In some embodiments, the nutrient medium can include at least one jasmonate production elicitor. In certain embodiments, the nutrient medium can include at least one fungal quorum sensing molecule and at least one jasmonate production elicitor. In some embodiments, the nutrient medium can include two or more fungal quorum sensing molecules. In some embodiments, the nutrient medium can include two or more jasmonate production elicitors. The use of two or more fungal quorum sensing molecules, or the use of two or more jasmonate production elicitors, or the combined use of at least one fungal quorum sensing molecule and at least one jasmonate production elicitor, may generate a synergistic effect on jasmonate production at higher titers.

Examples of fungal quorum sensing molecules suitable for use according to the present teachings include, but are not limited to, farnesol, tyrosol, tryptophol, γ-heptalactone, farnesoic acid, 1-phenyl-ethanol, 2-phenylethanol, multicolanic acid, multicolosic acid, multicolic acid, bytyrolactone-I, γ-butyrolactone, alpha-(1,3)-glucan, a-factor pheromone, alpha-factor pheromone, 3-octanone, 3-octanol, and 1-octen-3-ol. Preferred fungal quorum sensing molecules include farnesol, tyrosol, tryptophol, and γ-heptalactone. In certain embodiments, the nutrient medium may include farnesol, tyrosol, or both. In typical embodiments, the fungal quorum sensing molecule(s) may be present in the nutrient medium at a concentration of about 10-500 mg/L.

Examples of jasmonate production elicitors suitable for use according to the present teachings include, but are not limited to, various plant hormones, oxidative stressors, and histone deacetylase inhibitors. Representative plant hormones include, but are not limited to, ethylene (ET), indole-3-acetic acid (IAA), salicylic acid (SA), acetylsalicylic acid (ASA). Also included are various auxins, for example 4-chloroindole-3-acetic acid (4-Cl-IAA), 2-phenylacetic acid (PAA), indole-3-butyric acid (IBA), and indole-3-propionic acid (IPA), and gibberellins such as gibberellin A1 (GA1), gibberellic acid (GA3), ent-gibberellane, and ent-kaurene. In some preferred embodiments, the jasmonate production elicitor is a plant defense hormone such as abscisic acid (ABA).

In some embodiments, the oxidative stressor may be a reactive oxygen species (ROS) that is added to the nutrient medium. Typical reactive oxygen species include hydrogen peroxide, peroxide salts, peroxy acids, and superoxide salts. Oxidative stress may also be induced by the addition of organic compounds known to be redox-active. Viologens are a well-known family of redox-active heterocycles, and the viologen paraquat (methyl viologen, or MV) is widely used to induce oxidative stress by a mechanism believed to act as a superoxide generator to produce ROS through interactions with Complex I within the inner mitochondrial matrix.

Representative histone deacetylase inhibitors include, but are not limited to, valproic acid (VA) and sodium butyrate. Typically, the jasmonate production elicitor(s) is present in the nutrient medium at a concentration of about 10-500 mg/L.

In various embodiments, the nutrient medium may include at least one carbon source and at least one nitrogen source. Examples of a suitable carbon source include, but are not limited to, sucrose, starch, maltose, glucose, and fructose. The nitrogen source may either be an organic nitrogen source, an inorganic nitrogen source, or both. Examples of an organic nitrogen source include, but are not limited to, beef extract, peptone, corn pulp, yeast extract, and malt extraction. Examples of an inorganic nitrogen source include, but are not limited to, sodium nitrate, potassium nitrate, urea, and ammonium nitrate.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

FIG. 1 shows the chemical structures of jasmonic acid (JA) and its methyl ester, methyl jasmonate (MeJA).

FIG. 2 shows the morphologies of fungal strains Lasiodiplodia iranensis grown under different culture conditions: (Panel A) shaking at 250 rpm; (Panel B) stationary. The temperature was 30° C. for both conditions.

FIG. 3 shows the chemical structures of exemplary fungal quorum sensing molecules that may be used according to the present teachings, specifically: farnesol, tyrosol, tryptophol, and γ-heptalactone.

FIG. 4 shows HPLC (top left) and UV (top right) spectra of a JA standard from Sigma-Aldrich (MO, USA) and those (bottom left and bottom right, respectively) of ethyl acetate extracts of the fungal culture that produced JA. The JA standard was prepared in methanol at 1 g/L concentration.

FIG. 5 shows the effect of farnesol on the morphology and JA production level of the fungal cultures under shaken conditions 9 days after inoculation. CK stands for the control culture with 0.1% ethanol added to the medium; “Far” stands for the culture with 100 mg/L farnesol added. The picture on the left shows the different morphologies between the control and the farnesol-induced cultures. The graph on the right shows that the farnesol-induced culture achieved a JA production titer of over 300 mg/L.

FIG. 6 shows the effect of tyrosol on the morphology and JA production level of the fungal cultures under shaken conditions 7 days after inoculation. CK3 stands for the control culture with 0.1% ethanol added to the medium; “Tyr3” stands for the culture with 100 mg/L tyrosol added. The picture on the left shows the different morphologies between the control and the tyrosol-induced cultures. The graph on the right shows that the tyrosol-induced culture achieved a JA production titer of over 300 mg/L.

FIG. 7 shows the chemical structures of various jasmonate production elicitors that can be used according to the present teachings, including representative plant hormones, oxidative stressors, and histone deacetylase inhibitors.

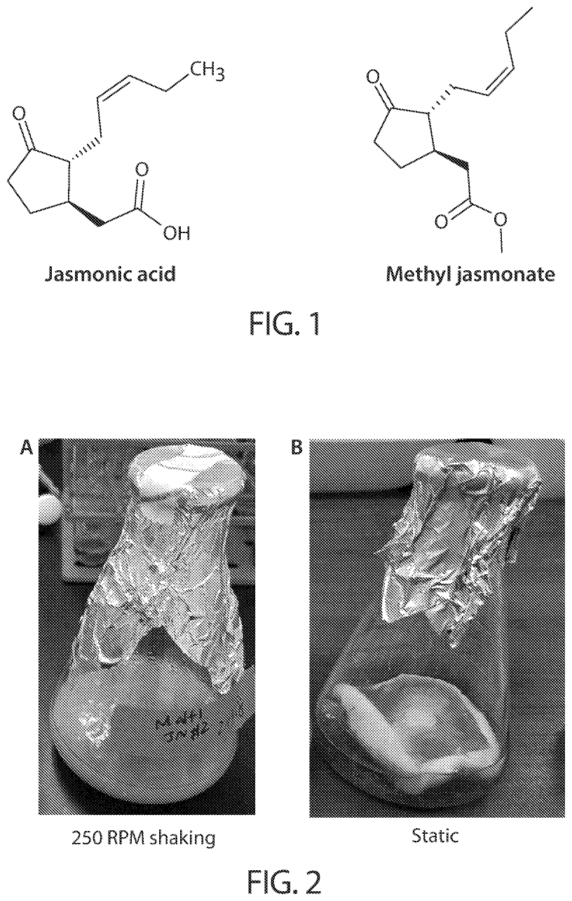

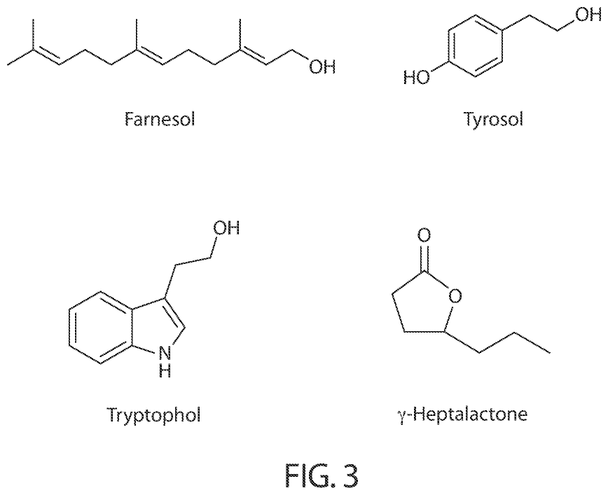

### DETAILED DESCRIPTION

The present teachings relate to methods for producing one or more jasmonates. The present methods generally involve cultivating a strain of filamentous fungus organism in a nutrient medium under agitation, and isolating a jasmonate product from the nutrient medium. The jasmonate(s) may be selected from jasmonic acid, methyl jasmonate, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-iso-jasmonic acid, cucurbic acid, 6-epi-cucurbic acid, 6-epi-cucurbic-acid-lactone, 12-hydroxy-jasmonic acid, 12-hydroxy-jasmonic-acid-lactone, 11-hydroxy-jasmonic acid, 8-hydroxy-jasmonic acid, homo-jasmonic acid, dihomo-jasmonic acid, 11-hydroxy-dihomo-jasmonic acid, 8-hydroxy-dihomo-jasmonic acid, tuberonic acid, tuberonic acid-O-beta-glucopyranoside, cucurbic acid-O-beta-glucopyranoside, 5,6-didehydrojasmonic acid, 6,7-didehydrojasmonic acid, 7,8-didehydrojasmonic acid, methyldihydroisojasmonate, amino acid conjugates of jasmonic acid, and the lower alkyl esters, salts, and stereoisomers thereof.

In the broadest sense, the nutrient medium according to the present teachings includes at least one fungal quorum sensing molecule or at least one jasmonate production elicitor, at least one carbon source, and at least one nitrogen source. As demonstrated by the experimental results included herein, the inventors have unexpectedly found that the addition of one or more fungal quorum sensing molecules and/or jasmonate production elicitors in the nutrient medium allows favorable fungal morphology to form even under agitation conditions. The formation of variable fungal morphology (mycelial mat) was shown to enhance jasmonate production in filamentous fungi such as Lasiodiplodia iranensis.

Quorum sensing (QS) is a method of communication between microbes that enables the coordination of group-based behavior based on population density, which relies on the production and release of small diffusible chemical signaling molecules in the extracellular environment (Mehmood et al., Molecules 2019 May; 24(10): 1950). Quorum sensing was first reported in the marine bacterium Alivibrio fischeri (Nealson et al. (1970) J. Bacteriol. 104, 313-322). Farnesol was the first fungal quorum sensing molecule discovered in the dimorphic fungus Candida albicans (Hornby, et al. (2001) Appl. Environ. Microbiol. 67, 2982-2992).

So far, many fungal quorum sensing molecules have been identified which include farnesol, tyrosol, tryptophol and γ-heptalactone. Multicolic acid, another fungal quorum sensing molecule has been reported to improve sclerotiorin production in Penicillium sclerotiorum (J Biotechnol. 2010 Jul. 20; 148(2-3):91-8). γ-Heptalactone was shown to regulate growth and secondary metabolite production in Aspergillus nidulans (Williams et al., Appl. Microbiol Biotechnol. 2012 November; 96(3):773-81). In addition, farnesol has been shown to induce morphological transition in the dimorphic fungus Ophiostoma piceae and higher extracellular esterase activities (De Salas et al., Appl Environ Microbiol. 2015 July; 81(13):4351-7).

However, the biological activities of quorum sensing molecules can be quite diverse. For example, while farnesol blocks yeast-to-filamentous transition at high cell density and promotes dispersal of yeast cells by inhibiting germ tube/hyphae formation, tyrosol stimulates hyphae production during the early stages of biofilm development and promotes germ tube formation (Padder et al., Microbiol Res. 2018 May; 210:51-58). Despite their different biological activities, both farnesol and tyrosol unexpectedly were found to show similar effects on morphology changes and jasmonic acid production in JA-producing filamentous fungi.

Fungal quorum sensing molecules that may be used according to the present teachings include, but are not limited to, one or more of farnesol, tyrosol, tryptophol, γ-heptalactone, farnesoic acid, 1-phenyl-ethanol, 2-phenylethanol, multicolanic acid, multicolosic acid, multicolic acid, butyrolactone-I, γ-butyrolactone, alpha-(1,3)-glucan, a-factor pheromone, alpha-factor pheromone, 3-octanone, 3-octanol, and 1-octen-3-ol. Preferred fungal quorum sensing molecules include farnesol, tyrosol, tryptophol, and γ-heptalactone. In certain embodiments, the nutrient medium can include farnesol, tyrosol, or both. Typically, the fungal quorum sensing molecule(s) may be present in the nutrient medium at a concentration of about 10-500 mg/L.

Jasmonate production elicitors are small molecules capable of inducing or awaking a cryptic biosynthetic pathway; in this instance, one involved in JA production by a filamentous fungus such as Lasiodiplodia iranensis. Examples of jasmonate production elicitors suitable for use according to the present teachings include, but are not limited to, various plant hormones, oxidative stressors, and histone deacetylase inhibitors. Representative plant hormones include, but are not limited to, ethylene (ET), indole-3-acetic acid (IAA), salicylic acid (SA), acetylsalicylic acid (ASA). Also included are various auxins, for example 4-chloroindole-3-acetic acid (4-Cl-IAA), 2-phenylacetic acid (PAA), indole-3-butyric acid (IBA), and indole-3-propionic acid (IPA), and gibberellins such as gibberellin A1 (GA1), gibberellic acid (GA3), ent-gibberellane, and ent-kaurene. In some preferred embodiments, the jasmonate production elicitor is a plant defense hormone such as abscisic acid (ABA).

In exemplary embodiments, the oxidative stressor may be a reactive oxygen species (ROS) that is added to the nutrient medium. Typical reactive oxygen species include hydrogen peroxide, peroxide salts, peroxy acids, and superoxide salts. Oxidative stress may also be induced by the addition of organic compounds known to be redox-active. Viologens are a well-known family of redox-active heterocycles, and the viologen paraquat (methyl viologen, or MV) is widely used to induce oxidative stress by a mechanism believed to act as a superoxide generator to produce ROS through interactions with Complex I within the inner mitochondrial matrix.

Representative histone deacetylase inhibitors include, but are not limited to, valproic acid (VA) and sodium butyrate. Typically, the jasmonate production elicitor(s) is present in the nutrient medium at a concentration of about 10-500 mg/L.

In various embodiments, the present methods can be conducted in a batch or continuous mode of operation. In a batch fermentation, the nutrient medium, culture and substrate, are combined and fermented until the jasmonate product becomes constant. In a continuous process, the substrate in the nutrient medium may be continuously recirculated through a fermentation reactor, with the provision that substrate and product are respectively added and removed from the recirculating medium.

In carrying out the present process, cultivation and fermentative incubation of the fungal strain are accomplished in an aqueous medium in the presence of usual nutrient substances (carbon source, nitrogen source, inorganic salts and growth factors) in addition to the one or more fungal quorum sensing molecules and/or jasmonate production elicitors. Examples of inorganic salts that can be included in the nutrient medium include, but are not limited to, the phosphate and/or sulfate salts of sodium, calcium, magnesium, and potassium. Additional nutrients also may be added, such as one or more B vitamins, one or more trace minerals such as iron, manganese, cobalt, copper, zinc, etc., as known by those skilled in the art. Fungal growth hormones such as 10-oxo-trans-8-decenoic acid and hercynine also may be included in the nutrient medium.

In a typical process, the filamentous fungus organism is first cultivated in inoculum quantities to produce a mature culture in a nutrient medium. The culture is inoculated into a fermenter nutrient medium and allowed to establish itself. The substrate is then added, and fermentation is continued until a steady concentration of the jasmonate product is present.

The cultivation and fermentative incubation of the filamentous fungus organism can be carried out under agitation at about 150 rpm to about 1500 rpm. The cultivation temperature can be between about 20° C. and about 35° C. Cultivation and incubation can proceed under aerobic conditions in a pH range of from about 4.5 up to about 9, preferably at 6. The jasmonate product can be isolated after at least 2 days of cultivation following addition of the substrate.

In various embodiments, the jasmonate product can be isolated from the nutrient medium by extraction with an extraction solvent such as ethyl acetate to form a jasmonate extract. The extraction solvent can be stripped to provide a concentrated jasmonate extract. Jasmonic acid present in the jasmonate extract can be converted to methyl jasmonate by esterification using methyl alcohol. The resulting methyl jasmonate can be further concentrated using techniques known by those skilled in the art. For example, fractionation can be performed, e.g., with silica gel, to separate different isomers.

Jasmonates such as jasmonic acid and methyl jasmonate produced according to the present teachings can be used for various applications in agriculture, food, fragrances, and medicine. For example, jasmonic acid has been tested as a natural pest control tool for crop plants against herbivores. Methyl jasmonate can be used as a food and flavor ingredient in products such as perfumes, personal care products, household care products, oral consumable products, and so forth. In addition, methyl jasmonate also has great potential to be developed for pharmaceutical uses, given its reported antidepressant, anti-aggressive, and anti-inflammatory effects.

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these examples, while indicating preferred embodiments of the subject technology, are given by way of illustration only. From the above discussion and these examples, one skilled in the art can ascertain the essential characteristics of the subject technology, and without departing from the spirit and scope thereof, can make various changes and modifications of the subject technology to adapt it to various uses and conditions.

### EXAMPLES

### Example 1: Morphologies of Lasiodiplodia Iranensis Under Different Culture Conditions

Cultures of Lasiodiplodia iranensis DWH-2 (deposited under CCTCC Deposit No. M2017288) were grown under two different conditions for the production of jasmonic acid (JA). One culture was grown in a shaker under agitation conditions (250 rpm). Another culture was grown in an incubator for stationary growth.

As shown in FIG. 2, under shaking conditions, fungal mycelia were freely dispersed with a porridge-like consistency throughout the culture (i.e., they formed mycelial clumps). In contrast, under stationary conditions, fungal mycelia aggregated into a mat.

Further, HPLC analysis demonstrated that under shaking conditions, little or no JA was produced, whereas under stationary conditions, the culture produced JA at titers of about 1 g/L similar to what was reported in Chinese Patent No. CN107227264B.

In light of the above results, specifically with regard to fungal morphology, it was demonstrated that JA production is correlated with mycelial aggregation in a filamentous fungus such as Lasiodiplodia iranensis.

### Example 2: Effects of Adding Farnesol to JA Production by Lasiodiplodia Iranensis

Cultures of Lasiodiplodia iranensis DWH-2 (deposited under CCTCC Deposit No. M2017288) were maintained on potato dextrose agar (PDA, from MilliporeSigma, Mo., USA) plates in an incubator at 30° C.

A square piece (˜1 cm×1 cm) of the PDA plate containing L. iranensis culture was cut and used to inoculate 50 ml of a nutrient medium with (Far) or without (CK) farnesol.

Specifically, farnesol (purchased from Sigma-Aldrich, Mo., USA) was dissolved in 70% ethanol to make a stock solution with a concentration of 100 g/L. The final working concentration in the culture medium is 100 mg/L (1,000 times dilution) for the sample containing farnesol. The nutrient medium contained the following: glucose (50 g/L); KNO₃ (8.9 g/L); KH₂PO₄ 2.0 (g/L); KCl 0.3 (g/L); MgSO₄.7H₂O 0.6 (g/L); FeSO₄.7H₂O (0.6 g/L); ZnSO₄.7H₂O (0.03 g/L); MnSO₄.7H₂O (0.003 g/L); CuSO₄.7H₂O (0.003 g/L); Na₂MoO₄.2H₂O 0.003 (g/L); and yeast extract (1.0 g/L).

The flasks were placed into a shaker set at 250 rpm and 30° C. After nine days of cultivation, extracts were taken and their JA contents were analyzed by HPLC.

For porridge-like cultures, 0.5 ml of the whole culture was taken as sample for further analysis. For mat-like cultures, 0.5 ml of the supernatant was used. To each sample, 10 μl of 2N HCl was added for acidification followed by adding 0.5 ml of ethyl acetate for JA extraction. After shaking at room temperature for 30 min, the samples were centrifuged at 15,000 rpm for 15 min. The ethyl acetate phase was used for HPLC analysis.

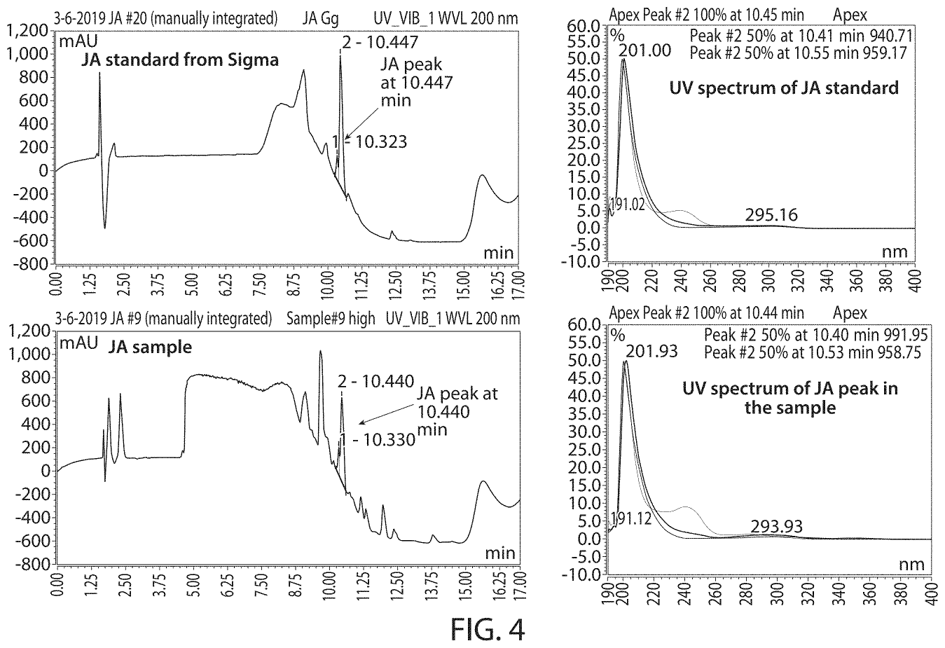

The HPLC was performed on Thermo Scientific Dionex Ultimate 3000 using an Acclaim™ 120, C18 column (3 μm 120A, 3×150 mm). The mobile phases were: A, 0.1% TFA (trifluoroacetic acid) and B, acetonitrile with the gradient: 0-5 min, 5% B; 5-9 min, 5-80% B; 9-13 min, 80% B; 13-14 min, 80-5% B; 14-17 min, 5% B. The detector wavelength for JA was 200 nm. FIG. 4 confirms that JA from the fungal cultures has the same retention time and UV spectrum as the JA standard from Sigma-Aldrich (MO, USA).

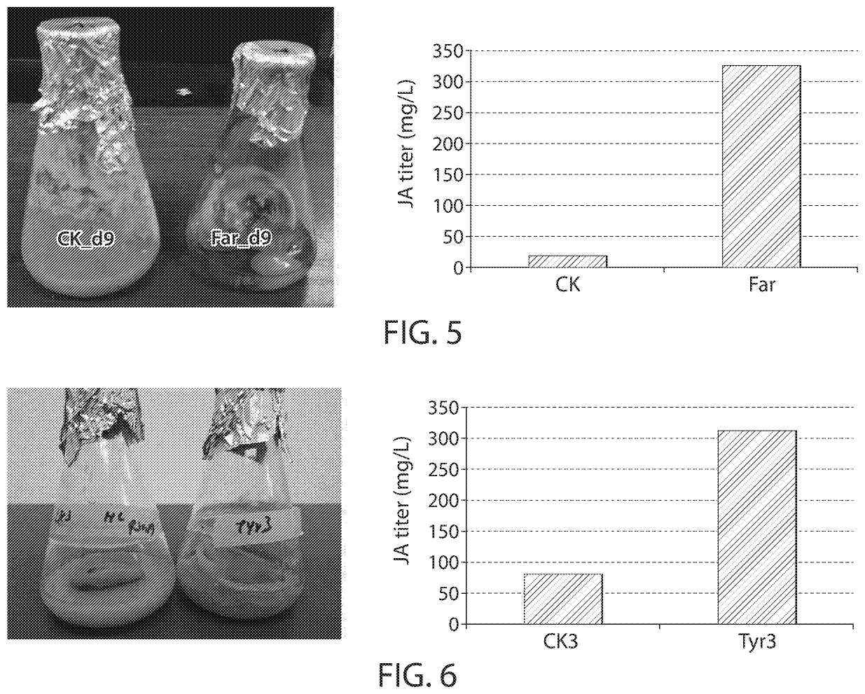

The effects of adding farnesol on JA production under shaking conditions were demonstrated in FIG. 5. As shown, the culture without the supplement of farnesol exhibited porridge-like morphology and produced little jasmonic acid (ca. 19 mg/L) under shaking conditions. In contrast, farnesol induced mycelial aggregation in L. iranensis, which resulted in the formation of mat-like microbial community despite shaking conditions. The sample with farnesol added was able to produce jasmonic acid at a titer of ˜327 mg/L.

Thus, these results confirmed that the addition of farnesol could be used to control the fungal morphology of filamentous fungi such as Lasiodiplodia iranensis. Specifically, Lasiodiplodia iranensis was able to form a mycelial mat under shaking conditions when farnesol was added. Given a mat-like morphology appears to be crucial for jasmonic acid biosynthesis by filamentous fungi, the addition of farnesol led to JA production at higher titers under shaking conditions.

### Example 3: Effects of Adding Tyrosol to JA Production by Lasiodiplodia Iranensis

The procedures described in Example 2 were repeated with tyrosol instead of farnesol. Specifically, a square piece (about 1 cm×1 cm in area) of the PDA plate containing L. iraniensis culture was cut and used to inoculate 30 ml of the same nutrient medium with (Tyr3) or without (CK3) tyrosol. Tyrosol (purchased from Sigma-Aldrich, Mo., USA) was dissolved in 70% ethanol to make a stock solution with a concentration of 100 g/L. The final working concentration in the culture medium is 100 mg/L (1,000 times dilution) for the sample containing tyrosol.

The flasks were placed into a shaker set at 250 rpm and 30° C. After seven days of cultivation, extracts were taken and their JA contents were analyzed by HPLC.

The effects of adding tyrosol on JA production under shaking conditions were demonstrated in FIG. 6. As shown, the culture without the supplement of tyrosol exhibited porridge-like morphology and produced little jasmonic acid (ca. 81 mg/L) under shaking conditions. In contrast, tyrosol induced mycelial aggregation in L. iranensis, which resulted in the formation of mat-like microbial community despite shaking conditions. The sample with tyrosol added was able to produce jasmonic acid at a titer of ˜314 mg/L.

Thus, these results confirmed that the addition of tyrosol could be used to control the fungal morphology of filamentous fungi such as Lasiodiplodia iranensis. Specifically, Lasiodiplodia iranensis was able to form a mycelial mat under shaking conditions when tyrosol was added. Given a mat-like morphology appears to be crucial for jasmonic acid biosynthesis by filamentous fungi, the addition of tyrosol led to JA production at higher titers under shaking conditions.

### Example 4: Effects of Adding One or More Jasmonate Production Elicitors to JA Production by Lasiodiplodia Iranensis

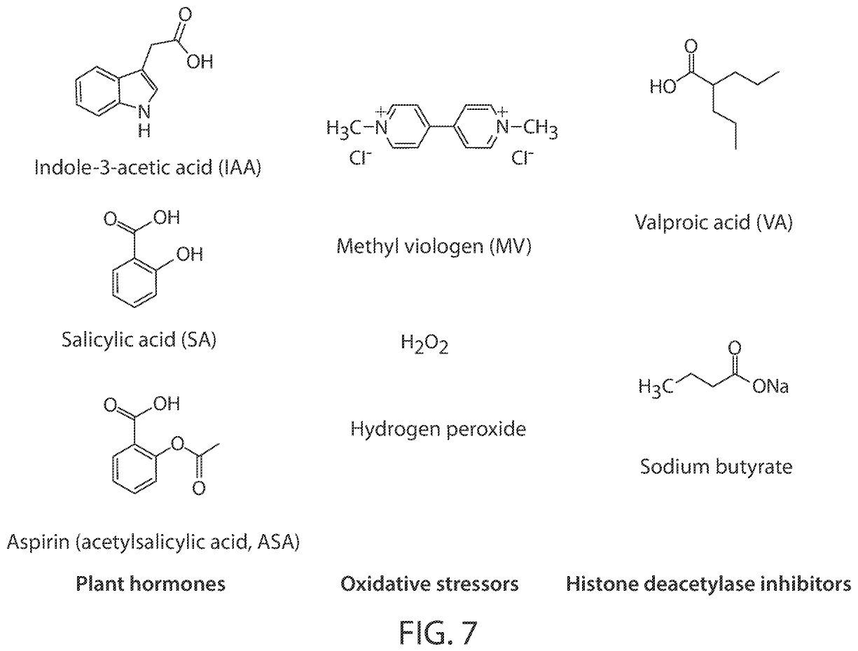

The procedures described in Example 2 were repeated with a jasmonate production elicitor instead of farnesol. Specifically, a square piece (about 1 cm×1 cm in area) of the PDA plate containing L. iraniensis culture was cut and used to inoculate 50 ml of the same nutrient medium with or without a jasmonate production elicitor. Jasmonate production elicitors are small molecules capable of inducing or awaking a cryptic biosynthetic pathway; in this case, one involved in JA production by a filamentous fungus such as Lasiodiplodia iranensis. Typical jasmonate production elicitors may be a plant hormone, an oxidative stressor, a histone deacetylase inhibitor, or an antibiotic. FIG. 7 shows the chemical structures of various jasmonate production elicitors that can be used according to the present teachings, including representative plant hormones such as indole-3-acetic acid (IAA), salicylic acid (SA), acetylsalicylic acid (ASA); representative oxidative stressors such as methyl viologen (MV), and hydrogen peroxide H₂O₂; and representative histone deacetylase inhibitors such as valproic acid (VA) and sodium butyrate.

Indole-3-acetic acid sodium salt (IAA), salicylic acid sodium salt (SA), acetylsalicylic acid (ASA), sodium butyrate, valproic acid sodium salt (VA), and hydrogen peroxide were purchased from Sigma-Aldrich (MO, USA). Stock solutions of sodium salts and H₂O₂ were prepared in water whereas the stock solution of ASA was prepared in 70% ethanol. The final working concentrations in the culture medium were 200 mg/L for IAA and sodium butyrate; 100 mg/L for SA and ASA; 10 mg/L for VA and 2 mM for H₂O₂.

The flasks were placed into a shaker set at 250 rpm and 30° C. After eight days of cultivation, extracts were taken and their JA contents were analyzed by HPLC.

In the control (i.e., without a jasmonate production elicitor), less than 50 mg/L of JA was produced. Each of the cultures with an added jasmonate production elicitor produced JA at a significantly higher titer, as summarized in Table 1 below.

TABLE 1
Production of JA with or without a jasmonate production elicitor

| Elicitor | Titer (mg/L) |
|---|---|
| Control | 50 |
| IAA | 400 |
| SA | 150 |
| ASA | 100 |
| Sodium butyrate | 175 |
| VA | 350 |
| H₂O₂ | 160 |

1. A method for producing a jasmonate, the method comprising:
cultivating a strain of a filamentous fungus organism in a nutrient medium under agitation, wherein the nutrient medium comprises at least one fungal quorum sensing molecule or at least one jasmonate production elicitor, at least one carbon source, and at least one nitrogen, source; and
isolating a jasmonate product from the nutrient medium.

2. The method according to claim 1, wherein the filamentous fungus organism is of the genus Lasiodiplodia.

3. The method according to claim 1, wherein the filamentous fungus organism is a strain of Lasiodiplodia iranensis.

4. The method of claim 3, wherein the strain of Lasiodiplodia iranensis organism is Lasiodiplodia iranensis DWH-2 deposited under CCTCC Deposit No. M2017288.

5. The method of claim 1, wherein the fungal quorum sensing molecule is selected from the group consisting of farnesol, tyrosol, tryptophol, and γ-heptalactone.

6. The method of claim 1, wherein the fungal quorum sensing molecule is selected from the group consisting of farnesoic acid, 1-phenyl-ethanol, 2-phenylethanol, multicolanic acid, multicolosic acid, multicolic acid, bytyrolactone-I, γ-butyrolactone, alpha-(1,3)-glucan, a-factor pheromone, alpha-factor pheromone, 3-octanone, 3-octanol, and 1-octen-3-ol.

7. The method of claim 1, wherein the nutrient medium comprises two or more fungal quorum sensing molecules selected from the group consisting of farnesol, tyrosol, tryptophol, γ-heptalactone, farnesoic acid, 1-phenyl-ethanol, 2-phenylethanol, multicolanic acid, multicolosic acid, multicolic acid, bytyrolactone-I, γ-butyrolactone, alpha-(1,3)-glucan, a-factor pheromone, alpha-factor pheromone, 3-octanone, 3-octanol, and 1-octen-3-ol.

8. The method of claim 1, wherein the nutrient medium comprises at least one of farnesol or tyrosol.

9. The method of claim 1, wherein the jasmonate production elicitor is selected from the group consisting of a plant hormone, an oxidative stressor, and a histone deacetylase inhibitor.

10. The method of claim 1, wherein the jasmonate production elicitor is selected from the group consisting of indole-3-acetic acid, salicylic acid, acetylsalicylic acid, methyl viologen, hydrogen peroxide, valproic acid, and sodium butyrate.

11. The method of claim 1, wherein the nutrient medium comprises two or more jasmonate production elicitors selected from the group consisting of indole-3-acetic acid, salicylic acid, acetylsalicylic acid, methyl viologen, hydrogen peroxide, valproic acid, and sodium butyrate.

12. The method of claim 1, wherein the nutrient medium comprises at least one fungal quorum sensing molecule and at least one jasmonate production elicitor.

13. The method of claim 1 wherein the carbon source is selected from the group consisting of sucrose, starch, maltose, glucose, and fructose.

14. The method of claim 1, wherein the nitrogen source is an organic nitrogen source selected from the group consisting of beef extract, peptone, corn pulp, yeast extract, and malt extraction.

15. The method of claim 1, wherein the nitrogen source is an inorganic nitrogen source selected from the group consisting of sodium nitrate, potassium nitrate, urea, and ammonium nitrate.

16. The method of claim 1, where the agitation is performed between about 150 rpm and about 1500 rpm.

17. The method of claim 1, wherein the cultivating step is performed at a temperature between about 20° C. and about 35° C.

18. (canceled)

19. The method of claim 1, wherein the at least one fungal quorum sensing molecule is present in the nutrient medium at about 10-500 mg/L.

20. The method of claim 1, wherein the at least one jasmonate production elicitor is present in the nutrient medium at about 10-500 mg/L if the jasmonate production elicitor is a solid.

21. The method of claim 1, wherein the nutrient medium further comprises a fungal growth hormone selected from the group consisting of 10-oxo-trans-8-decenoic acid and hercynine.

22. (canceled)
